(19) United States
(12) Patent Application Publication
(10) Pub. No.: US 2005/0095705 A1
(43) Pub. Date: May 5, 2005

(54) Method for production of oncolytic adenoviruses

(76) Inventors: Michael Kadan, Adamstown, MD (US); Ronald Kaptur, Manhattan, KS (US); David Brousseau, San Francisco, CA (US); Denice Mittelstaedt, San Diego, CA (US); Yuanhao Li, Palo Alto, CA (US)

Correspondence Address: Supervisor, Patent Prosecution Services;PIPER RUDNICK LLP,, 1200 Nineteenth Street, N.W., Washington, DC, 20036-2412, US

(21) Appl. No.: 10/824,796

(22) Filed: Apr. 14, 2004

Related U.S. Application Data
(60) Provisional application No. 60/463,143, filed on Apr. 15, 2003.

(51) Int. Cl. C12N 5/08; C12N 15/86

(52) U.S. Cl. 435/367; 435/456

(57) ABSTRACT

HeLa-S3 cells comprising replication-competent adenovirus vectors are provided. Also provided are HeLa-S3 producer cell lines and methods for producing replication-competent adenovirus using the same.

This application is related to Provisional U.S. Patent Application Ser. No. 60/463,143, filed Apr. 15, 2003, which is incorporated herein by reference in its entirety.

### FIELD OF THE INVENTION

The present invention generally relates to the production of oncolytic adenoviruses. More particularly, the present invention relates to the use of the HeLa-S3 cell line for the production of oncolytic adenoviruses.

### BACKGROUND OF THE INVENTION

Adenoviruses form the basis of some of the most innovative and potentially powerful disease-fighting tools. One such tool is gene therapy, in which an exogenous nucleotide sequence provided to a cell. This approach holds great potential in treating not only cancer, but many other diseases as well, including cystic fibrosis, anemia, hemophilia, diabetes, Hungtington's disease, AIDS, abnormally high serum cholesterol levels, certain immune deficiencies, and many forms of cancer. Gene therapy generally relies upon a delivery vehicle, such as a viral vector in order to provide the exogenous sequence to a cell. Recombinant adenovirus has shown some therapeutic efficacy against these diseases. For reviews, see Kim et al. (1996) Mol. Med. Today 12:519-527 and Smith et al. (1996) Gene Therapy 3:496-502. Adenoviruses that replicate selectively in target cells are being developed as therapeutic agents for treatment of cancer.

Helper virus-independent production of adenovirus can require a packaging cell line that complements for viral gene products. Adenovirus of interest, including oncolytic adenovirus, conditionally replicative adenovirus, and replication defective adenovirus are frequently engineered to have genetic modifications in the E1 early gene region (genetic map units 1.30 to 9.24) of the virus genome. Typical modifications include deletions within the E1 gene region and/or replacement of the E1A promoter with a tissue-specific promoter, e.g. myosin light chain, keratin, PKG, etc.

Replication-competent viral vectors have been developed for which selective replication in cancer cells preferentially destroys those cells. Various cell-specific replication-competent adenovirus constructs, which preferentially replicate in (and thus destroy) certain cell types, are described in, for example, WO 95/19434, WO 96/17053, WO 98/39464, WO 98/39465, WO 98/39467, WO 98/39466, WO 99/06576, WO 99/25860, WO 00/15820, WO 00/46355, WO 02/067861, WO 02/06862, U.S. Patent application publication US 20010053352 and U.S. Pat. Nos. 5,698,443, 5,871,726, 5,998,205, and 6,432,700. Replication-competent adenovirus vectors have been designed that specifically replicate in tumor cells.

Historically, adenovirus vectors were attenuated for replication by removal of the E1 gene region. Because this function is essential for viral replication, a cell line expressing this gene was necessary for the propagation of these attenuated viruses.

Available packaging cell lines typically contain Ad genes that have been deleted from the vector but are required for viral replication. In some cases overlapping sequences between the host cell and adenoviral vector are not completely eliminated. For example, the human embryonic kidney derived 293 cells (Graham et al. (1977) J. General Virology 36:59-74) have been widely used for propagating Adenoviral vectors. However, due to substantial overlapping sequences between the Adenoviral vector genome and the 293 cell line, recombination events occur that result in the generation of a replication competent adenoviral particles.

Improvements have been made to reduce the possibility of generating replication competent vectors due to recombination events between the packaging cell line and the vector via reduction in the sequences common to the vector and cell line (Fallaux et al. (1998) Human Gene Therapy 9:1909-1917). For example, U.S. Pat. No. 5,994,128 describes cell lines that complement for both E1A and/or E1B, while retaining the natural E1B promoter sequences. Studies performed using the PER.C6 cell line demonstrated that, despite a single region of homology between this cell line and the adenoviral vector, RCA were generated and cytopathic effects were observed in a cell based assay (Kim et al. (2001) Exp. Mol. Med 33(3)145-9). When analyzed, the RCA were shown to contain the PGK promoter-E1 gene, derived from the plasmid that was employed to construct the PER.C6 cell line. The same problem of residual sequence overlap is true of other cell lines developed as alternatives to 293 cells (see, for example, Massie et al., U.S. Pat. No. 5,891,690; Kovesdi et al., WO 95/34671, Kedan et al., PCT/US95/15947, Schiedner et al. (2002) Human Gene Therapy, 11:2105-2116).

A human tumor-derived cell line would potentially be useful for producing oncolytic adenoviruses. However, some oncolytic adenoviruses are designed to grow efficiently only in a very specific cancer cell type. Consequently, there remains the potential for unwanted recombination events between the cell line and the adenoviral vector.

### SUMMARY OF THE INVENTION

The present invention addresses the need for an additional cell line that can be used for the production on oncolytic adenoviruses by disclosing that the HeLa-S3 cell line overcomes the problems set forth above and is therefore ideal for the production on oncolytic adenoviruses.

In one aspect, the present invention provides a HeLa-S3 cell comprising a replication-competent adenovirus vector. The replication-competent adenovirus vector may have one or more of the following features:
(a) the replication-competent adenovirus vector may comprise a heterologous transcriptional regulatory element (TRE) sequence operatively linked to the coding region of a gene that is essential for replication, wherein the TRE functions in the cell so that replication of the vector occurs in the cell rendering the adenovirus vector tissue-specific or tumor-specific;
(b) the replication-competent adenovirus vector may comprise a mutation or deletion in the E1b gene, wherein the encoded E1b protein lacks the capacity to bind p53; and
(c) the replication-competent adenovirus vector may comprise a mutation or deletion in the E1a gene, wherein the encoded E1a protein lacks the capacity to bind RB.

The heterologous transcriptional regulatory element (TRE) may comprise a promoter or enhancer, examples of which include an E2F-responsive TRE, a human telomerase reverse transcriptase (hTERT) TRE, an osteocalcin TRE, a carcinoembryonic antigen (CEA) TRE, a DF3 TRE, an α-fetoprotein TRE, an ErbB2 TRE, a surfactant TRE, a tyrosinase TRE, a PRL-3 TRE, a MUC1/DF3 TRE, a TK TRE, a p21 TRE, a cyclin TRE, an HKLK2 TRE, a uPA TRE, a HER-2neu TRE, a prostate specific antigen (PSA) TRE, and a probasin TRE.

The replication-competent adenovirus vector in the HeLa-S3 cell may have an E1a, E1b, E2a, E2b or E4 coding region that is operatively linked to one or more TREs. When the replication-competent adenovirus vector comprises more than one TRE, the TREs may be different.

The replication-competent adenovirus vector in the HeLa-S3 cell may further comprise a heterologous gene, such as the gene encoding GM-CSF.

The invention further provides producer cell lines comprising the HeLa-S3 cells of the invention and methods of producing the same.

### DESCRIPTION OF THE FIGURES

FIG. 1 is a graphical presentation of the data in Table 1. It represents growth characteristics in CD293 media, showing seeding/harvest densities for a given portion of the cell culture's life span. The first 300-350 hours are during adaptation; therefore, during this stage cells are still adherent as compared to when the cells go into suspension approximately 400 hours post-thaw.

FIG. 2 is a graphical presentation of the data in Table 2. It represents doubling times in CD293 media, showing doubling times after the cells go into suspension. It does not include the adaptation stage. This data correlates roughly to hours 400 on Table 1 and FIG. 1.

FIG. 3 is a graphical presentation of the data in Table 3. It represents growth characteristics in EX-CELL™ 293 media, showing seeding/harvest densities for a given portion of the cell culture's life span. The first 300-350 hours are during adaptation; therefore, during this stage cells are still adherent as compared to when the cells go into suspension approximately 400 hours post-thaw.

FIG. 4 is a graphical presentation of the data in Table 4. It represents doubling times in EX-CELL™ 293 media, showing doubling times after the cells go into suspension. It does not include the adaptation stage. This data correlates roughly to hours 400 and on in Table 3 and FIG. 3.

FIG. 5 is a graphical presentation of the data in Table 5. It represents the thaw testing history of HeLa-S3 in EX-CELL™ 293 serum-free media. The data shows growth characteristics for HeLa-S3 suspension line adapted in EX-CELL™ 293 media, but frozen with and without 5% sucrose.

FIG. 6 shows a flow diagram for a purification process for Adenovirus produced in Hela-S3 cells.

FIG. 7 shows the results of a viral burst size for various adenoviruses grown in Hela-S3 cells.

FIG. 8 shows the results of viral growth kinetics on Hela-S3 cells.

### DETAILED DESCRIPTION OF THE INVENTION

The invention provides novel compositions comprising a HeLa-S3 cell which is serves as a vehicle for production of replication-competent adenovirus. The replication-competent adenovirus vectors may have a variety of features, further described in the summary of the invention and below.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

Definitions

Unless otherwise indicated, all terms used herein have the same meaning as they would to one skilled in the art and the practice of the present invention will employ, conventional techniques of microbiology and recombinant DNA technology, which are within the knowledge of those of skill of the art.

In describing the present invention, the following terms are employed and are intended to be defined as indicated below.

The term “HeLa-S3” means the human cervical tumor-derived cell line available from American Type Culture Collection (ATCC, Manassas, Va.) and designated as ATCC number CCL-2.2. HeLa-S3 is a clonal derivative of the parent HeLa line (ATCC CCL-2). HeLa-S3 was cloned in 1955 by T. T. Puck et al. (J. Exp. Med. 103: 273-284 (1956)).

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. Preferably, a vector of the invention comprises DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A nucleic acid sequence is “operatively linked” when it is placed into a functional relationship with another nucleic acid sequence. For example, a promoter or regulatory DNA sequence is said to be “operatively linked” to a DNA sequence that codes for an RNA or a protein if the two sequences are operatively linked, or situated such that the promoter or regulatory DNA sequence affects the expression level of the coding or structural DNA sequence. Operatively linked DNA sequences are typically, but not necessarily, contiguous.

The term “ORF” means Open Reading Frame.

As used herein, the terms “adenovirus” and “adenoviral particle” are used to include any and all viruses that may be categorized as an adenovirus, including any adenovirus that infects a human or an animal, including all groups, subgroups, and serotypes. Numerous adenovirus serotypes are currently available from ATCC and the invention contemplates the production of any serotype of adenovirus available from any source. The adenoviruses that can be produced according to the invention may be of human or non-human origin. For instance, an adenovirus can be of subgroup A (e.g., serotypes 12, 18, 31), subgroup B (e.g., serotypes 3, 7, 11, 14, 16, 21, 34, 35), subgroup C (e.g., serotypes 1, 2, 5, 6), subgroup D (e.g., serotypes 8, 9, 10, 13, 15, 17, 19, 20, 22-30, 32, 33, 36-39, 42-47), subgroup E (serotype 4), subgroup F (serotype 40, 41), or any other adenoviral serotype. Preferred serotypes are adenovirus serotypes 2(Ad2), 5 (Ad5) and 35 (Ad35).

Thus, as used herein, “adenovirus” and “adenovirus particle” refer to the virus itself or derivatives thereof and cover all serotypes and subtypes and both naturally occurring and recombinant forms, except where indicated otherwise. Preferably, such adenoviruses are ones that infect human cells. Such adenoviruses may be wildtype or may be modified in various ways known in the art or as disclosed herein. Such modifications include modifications to the adenovirus genome that is packaged in the particle in order to make an infectious virus. Such modifications include deletions known in the art, such as deletions in one or more of the E1a, E1b, E2a, E2b, E3, or E4 coding regions. Such modifications also include deletions of all of the coding regions of the adenoviral genome. Such adenoviruses are known as “gutless” adenoviruses. The terms also include replication-conditional adenoviruses; that is, viruses that preferentially replicate in certain types of cells or tissues but to a lesser degree or not at all in other types. In a preferred embodiment of the invention, the adenoviral particles replicate in abnormally proliferating tissue, such as solid tumors and other neoplasms. These include the viruses disclosed in U.S. Pat. Nos. 5,677,178, 5,698,443, 5,871,726, 5,801,029, 5,998,205, and 6,432,700, the disclosures of which are incorporated herein by reference in their entirety. Such viruses are sometimes referred to as “cytolytic” or “cytopathic” viruses (or vectors), and, if they have such an effect on neoplastic cells, are referred to as “oncolytic” viruses (or vectors).

The terms “adenovirus vector” and “adenoviral vector” are used interchangeably and are well understood in the art to mean a polynucleotide comprising all or a portion of an adenovirus genome. An adenoviral vector of this invention may be in any of several forms, including, but not limited to, naked DNA, DNA encapsulated in an adenovirus capsid, DNA packaged in another viral or viral-like form (such as herpes simplex, and AAV), DNA encapsulated in liposomes, DNA complexed with polylysine, complexed with synthetic polycationic molecules, conjugated with transferrin, complexed with compounds such as PEG to immunologically “mask” the molecule and/or increase half-life, or conjugated to a non-viral protein.

As used herein, the terms “vector,” “polynucleotide vector,” “polynucleotide vector construct,” “nucleic acid vector construct,” and “vector construct” are used interchangeably herein to mean any nucleic acid construct for gene transfer, as understood by those skilled in the art.

As used herein, the term “viral vector” is used according to its art-recognized meaning. It refers to a nucleic acid vector construct that includes at least one element of viral origin and may be packaged into a viral vector particle. The viral vector particles may be utilized for the purpose of transferring DNA, RNA or other nucleic acids into cells either in vitro or in vivo. For purposes of the present invention, the viral vector is preferably an adenoviral vector.

The terms “virus,” “viral particle,” “vector particle,” “viral vector particle,” and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring DNA into cells either in vitro or in vivo. For purposes of the present invention, these terms preferably refer to adenoviruses, including recombinant adenoviruses formed when an adenoviral vector of the invention is encapsulated in an adenovirus capsid.

The term as used herein “replication-competent” as used herein relative to the adenoviral vectors of the invention means the adenoviral vectors and particles preferentially replicate in certain types of cells or tissues but to a lesser degree or not at all in other types. In one embodiment of the invention, the adenoviral vector and/or particle selectively replicates in tumor cells and or abnormally proliferating tissue, such as solid tumors and other neoplasms. Such viruses may be referred to as “oncolytic viruses” or “oncolytic vectors” and may be considered to be “cytolytic” or “cytopathic” and to effect “selective cytolysis” of target cells. Preferred oncolytic adenoviruses produced according to the present invention use tumor-specific regulatory elements to control the expression of early viral genes essential for replication. See, e.g., WO 96/17053, WO 99/25860, WO 02/067861, WO 02/068627, WO 20004/009790; U.S. Pat. Nos. 5,677,178, 5,698,443, 5,871,726, 5,801,029, 5,998,205, 6,432,700, 6,692,736 and 6,495,130, and U.S. Patent Publication No. 2003-0068307 all of which are incorporated herein by reference. Such oncolytic adenoviruses will specifically replicate and lyse tumor cells if the gene that is essential for replication is under the control of a TRE that is tumor-specific.

By “pan-cancer” is meant that the replication-competent adenoviral vectors of the invention selectively replicate in tumor cells and or abnormally proliferating tissue in general and replication is not necessarily limited to a particular type of cancer.

“Preferential replication” and “selective replication” and “specific replication” may be used interchangeably and mean that the virus replicates more in a target cell than in a non-target cell. Preferably, the virus replicates at a significantly higher rate in target cells than non target cells; preferably, at least about 3 fold higher, more preferably, usually at least about 10-fold higher, it may be at least about 50-fold higher, and in some instances at least about 100-fold, 400-fold, 500-fold, 1000-fold or even 1e6 higher. In one embodiment, the virus replicates only in the target cells (that is, does not replicate at all or replicates at a very low level in non-target cells).

Other oncolytic adenoviruses produced according to the present invention have one or more mutations or deletions in the E1a and/or E1b genes such that the E1a protein lacks the capacity to bind RB and such that the E1b protein lacks the capacity to bind p53. (See, e.g., U.S. Pat. No. 5,677,178.)

The term “gene essential for replication” refers to a nucleic acid sequence whose transcription is required for a viral vector to replicate in a target cell. For example, in an adenoviral vector of the invention, a gene essential for replication may be one or more of the E1a, E1b, E2a, E2b, or E4 genes.

As used herein, a “packaging cell” is a cell that is able to package adenoviral genomes or modified genomes to produce viral particles. It can provide a missing gene product or its equivalent. Thus, packaging cells can provide complementing functions for the genes deleted in an adenoviral genome and are able to package the adenoviral genomes into the adenovirus particle. The production of such particles requires that the genome be replicated and that those proteins necessary for assembling an infectious virus are produced. The particles also can require certain proteins necessary for the maturation of the viral particle. Such proteins can be provided by the vector or by the packaging cell.

“Regulatory elements” are sequences involved in controlling the expression of a nucleotide sequence. Regulatory elements include promoters, enhancers, and termination signals. They also typically encompass sequences required for proper translation of the nucleotide sequence.

As used herein, a “transcriptional response element” or “transcriptional regulatory element”, or “TRE” is a polynucleotide sequence, preferably a DNA sequence, comprising one or more enhancer(s) and/or promoter(s) and/or promoter elements such as a transcriptional regulatory protein response sequence or sequences, which increases transcription of an operably linked polynucleotide in a host cell that allows a TRE to function.

The term “promoter” refers to an untranslated DNA sequence usually located upstream of the coding region that contains the binding site for RNA polymerase II and initiates transcription of the DNA. The promoter region may also include other elements that act as regulators of gene expression.

Promoters and other transcriptional regulatory elements (TREs) that are tumor-specific include, but are not limited to, an E2F responsive promoter such as the E2F-1 promoter, a human telomerase reverse transcriptase (hTERT) promoter, an osteocalcin promoter, a carcinoembryonic antigen (CEA) promoter, a DF3 promoter, an α-fetoprotein promoter, an ErbB2 promoter, a surfactant promoter, a tyrosinase promoter, a MUC1/DF3 promoter, a TK promoter, a p21 promoter, a cyclin promoter, an HKLK2 promoter, a uPA promoter, a HER-2neu promoter, a prostate specific antigen (PSA) promoter, and a probasin promoter.

The term “E2F promoter” as used herein refers to a native E2F promoter and functional fragments, mutations and derivatives thereof. The E2F promoter does not have to be the full-length or wild type promoter. One skilled in the art knows how to derive fragments from an E2F promoter and test them for the desired selectivity. An E2F promoter fragment of the present invention has promoter activity selective for tumor cells, i.e. drives tumor selective expression of an operatively linked coding sequence. A number of examples of E2F promoters are known in the art. See, e.g., Parr et al. Nature Medicine 1997:3(10) 1145-1149, WO 02/067861, US20010053352 and WO 98/13508.

The term “telomerase promoter” or “TERT promoter” as used herein refers to a native TERT promoter and functional fragments, mutations and derivatives thereof. The TERT promoter does not have to be the full-length or wild type promoter. One skilled in the art knows how to derive fragments from a TERT promoter and test them for the desired selectivity. A TERT promoter fragment of the present invention has promoter activity selective for tumor cells, i.e. drives tumor selective expression of an operatively linked coding sequence. In one embodiment, the TERT promoter of the invention is a mammalian TERT promoter. In another embodiment, the mammalian TERT promoter is a human TERT promoter. See, e.g., WO 98/14593 and WO 00/46355 for exemplary TERT promoters that find utility in the compositions and methods of the present invention.

The term “enhancer” within the meaning of the invention may be any genetic element, e.g., a nucleotide sequence that increases transcription of a coding sequence operatively linked to a promoter to an extent greater than the transcription activation effected by the promoter itself when operatively linked to the coding sequence, i.e. it increases transcription from the promoter.

The terms “coding sequence” and “coding region” refer to a nucleic acid sequence that is transcribed into RNA such as mRNA, rRNA, tRNA, snRNA, sense RNA or antisense RNA. In one embodiment, the RNA is then translated in a cell to produce a protein.

The term “expression” refers to the transcription and/or translation of an endogenous gene or a transgene in a cell. In the case of an antisense construct, expression may refer to the transcription of the antisense DNA only.

The term “gene” refers to a defined region that is located within a genome and that, in addition to the aforementioned coding sequence, comprises other, primarily regulatory, nucleic acid sequences responsible for the control of expression, i.e., transcription and translation of the coding portion. A gene may also comprise other 5′ and 3′ untranslated sequences and termination sequences. Depending on the source of the gene, further elements that may be present are, for example, introns.

The terms “heterologous” and “exogenous” as used herein with reference to nucleic acid molecules such as TREs, promoters and gene coding sequences, refer to sequences that originate from a source foreign to a particular virus or host cell or, if from the same source, are modified from their original form. Thus, a heterologous gene in a virus or cell includes a gene that is endogenous to the particular virus or cell but has been modified through, for example, codon optimization. The terms also include non-naturally occurring multiple copies of a naturally occurring nucleic acid sequence. Thus, the terms refer to a nucleic acid segment that is foreign or heterologous to the virus or cell, or homologous to the virus or cell but in a position within the host viral or cellular genome in which it is not ordinarily found.

The term “homologous” as used herein with reference to a nucleic acid molecule refers to a nucleic acid sequence naturally associated with a host virus or cell.

The term “native” refers to a gene that is present in the genome of wildtype virus or cell.

The term “naturally occurring” or “wildtype” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

As used herein, the terms “cancer”, “cancer cells”, “neoplastic cells”, “neoplasia”, “tumor”, and “tumor cells” (used interchangeably) refer to cells that exhibit relatively autonomous growth, so that they exhibit an aberrant growth phenotype or aberrant cell status characterized by a significant loss of control of cell proliferation. Neoplastic cells can be malignant or benign. It follows that cancer cells are considered to have an aberrant cell status.

An aberrant cell status is defined in relation to a cell of the same type, which is in a non-dividing/regulated dividing state and under normal physiological conditions.

Characteristics Of Adenovirus Vectors Produced Using Hela-S3 Cells

In one embodiment, adenoviruses produced according to the invention comprise an adenoviral nucleic acid backbone, wherein the nucleic acid backbone comprises in sequential order: a left ITR, a termination signal sequence, a tumor specific TRE that is operatively linked to a first gene essential for replication of the adenoviral vector, an adenoviral packaging signal, and a right ITR.

In another embodiment, adenoviruses produced according to the invention comprise an adenoviral nucleic acid backbone, wherein the nucleic acid backbone comprises in sequential order: a left ITR, an adenoviral packaging signal, a termination signal sequence, a tumor specific TRE that is operatively linked to a first gene essential for replication of the adenoviral vector, and a right ITR.

In yet another embodiment, adenoviruses produced according to the invention comprise an adenoviral nucleic acid backbone, wherein the nucleic acid backbone comprises in sequential order: a left ITR, a termination signal sequence, a first tumor specific TRE operatively linked to a first gene essential for replication of the adenoviral vector, a second TRE operatively linked to a second gene essential for replication, an adenoviral packaging signal, and a right ITR.

In a further embodiment, adenoviruses produced according to the invention comprise an adenoviral nucleic acid backbone, wherein the nucleic acid backbone comprises in sequential order: a left ITR, an adenoviral packaging signal, a termination signal sequence, a first tumor specific TRE operatively linked to a first gene essential for replication of the adenoviral vector, a second tumor specific TRE operatively linked to a second gene essential for replication, and a right ITR. The first and second tumor specific TREs may be essentially the same, derived from the same promoter(s) or may be derived from different promoters.

An adenovirus produced according to the invention may comprise a termination signal sequence. The termination signal sequence increases the therapeutic effect because it reduces replication and toxicity of the oncolytic adenovirus in non-target cells. Oncolytic adenoviruses with a polyadenylation signal inserted upstream of the E1a coding region have been shown to be superior to their non-modified counterparts as they have demonstrated the lowest level of E1a expression in nontarget cells. Thus, insertion of a polyadenylation signal sequence to stop nonspecific transcription from the left ITR improves the specificity of E1a expression from the respective TRE. Insertion of the polyadenylation signal sequences reduces replication of the oncolytic adenoviral vector in nontarget cells and therefore toxicity. A termination signal sequence may also be placed before (5′) any TRE in the vector. In one embodiment, the terminal signal sequence is placed before a heterologous TRE operatively linked to the E1b or E4 gene.

In another embodiment, an adenovirus produced according to the invention further comprises a deletion upstream of the termination signal sequence, such as a deletion between nucleotides 103 and 551 of the adenoviral type 5 backbone or corresponding positions in other serotypes. A deletion in the packaging signal 5′ to the termination signal sequence may be such that the packaging signal becomes non-functional. In one embodiment, the deletion comprises a deletion 5′ to the termination signal sequence wherein the deletion spans at least the nucleotides 189 to 551. In another embodiment the deletion comprises a deletion 5′ to the termination signal sequence wherein the deletion spans at least nucleotides 103 to 551 (FIG. 2 of WO 02/067861 and WO 02/068627). In this particular embodiment, it is preferred that the packaging signal is located (i.e. re-inserted) at a position 3′ to the termination signal sequence and downstream of the tumor specific TRE-driven gene essential for replication.

While any adenovirus vector may be produced using HeLa S3 cells and the methods described herein, a description of exemplary adenovirus vectors follows:

Ar17pAE2fFTrtex, which is described in detail in PCT/US02/05300 (WO 02/067861), is a tumor-specific oncolytic adenovirus designed for systemic delivery (e.g. IV) for the treatment of a broad range of cancer indications. The replication of Ar17pAE2fFTrtex is engineered to be dependent on the presence of the two most common alterations in human cancer, namely defects in the Rb-pathway (˜85% of all cancers) and over expression of telomerase (˜85% of all cancers).

Consistent with Ar6pAE2fE3F, Ar17pAE2fFTrtex utilizes the E2F-1 TRE to control expression of the adenoviral E1a gene. To increase tumor selectivity appropriate for systemic delivery, the adenoviral E4 gene in Ar17pAE2fFTrtex is controlled by a hTERT (human telomerase reverse transcriptase) TRE. Ar17pAE2fFTrtex is expected to replicate in the majority of cancer cells, leading to tumor specific-expression of toxic viral proteins, cytolysis, and enhancement of sensitivity to chemotherapy, cytokines and cytotoxic T lymphocytes.

CG5757 is a replication-competent adenovirus vector which comprises a human E2F TRE operatively linked to E1a and an hTERT TRE operatively linked to E1b wherein the E1b region comprises a deletion in the 19K coding region.

CG4030 is a replication-competent adenovirus vector which comprises an SV40 pA, a human E2F TRE operatively linked to E1a and an hTERT TRE linked to E4 and a relocated adenoviral packaging signal 5′ to the right ITR.

OSB029 is a replication-competent adenovirus vector that is similar to CG4030 except the adenovirus packaging signal is located in the wildtype position 3′ to left ITR.

OV947 is a replication-competent adenovirus vector which comprises a human E2F TRE operatively linked to E1a and an hTERT TRE operatively linked to E1b.

An adenovirus vector produced according to the invention may comprise a mutation or deletion in the E3 region. However, in an alternative embodiment, all or a part of the E3 region may be preserved or re-inserted. See, e.g., U.S. Pat. No. 6,495,130, incorporated herein by reference. Presence of all or a part of the E3 region may decrease the immunogenicity of the virus. It also may increase cytopathic effect in tumor cells and decrease toxicity to normal cells. Preferably, the virus expresses more than half of the E3 proteins.

In an alternative embodiment, an adenovirus produced according to the invention further comprises a mutation or deletion in the E1b gene. See U.S. Pat. No. 5,677,178. Preferably the mutation or deletion in the E1b gene is such that the E1b protein lacks the capacity to bind p53. This modification of the E1b region may be combined with viruses where all or a part of the E3 region is present.

In another alternative embodiment, an adenovirus produced according to the invention further comprises a mutation or deletion in the E1a gene. See U.S. Pat. No. 5,677,178. Preferably the mutation or deletion in the E1a gene is such that the E1a protein lacks the capacity to bind RB. This modification of the E1b region may be combined with viruses where all or a part of the E3 region is present.

In yet another embodiment, an adenovirus produced according to the invention further comprises at least one heterologous coding sequence, such as a therapeutic gene coding sequence. The therapeutic gene, preferably in the form of cDNA, can be inserted in any position that does not adversely affect the infectivity or replication of the virus. Preferably, it is inserted in the E3 region in place of at least one of the polynucleotide sequences coding for the E3 proteins. For example, the therapeutic gene may be inserted in place of the 19 kD or 14.7 kD E3 gene.

To further enhance therapeutic efficacy, the vectors of the invention may include one or more transgenes that have a therapeutic effect, such as enhancing cytotoxicity so as to eliminate unwanted target cells. The transgene may be under the transcriptional control of a cancer-specific TRE. The transgene may be regulated independently of the adenovirus gene regulation, e.g. having separate promoters, which may be the same or different, or may be coordinately regulated, e.g. having a single promoter in conjunction with an IRES or a self-processing cleavage sequence, such as a 2A sequence. In this approach expression of the E1A and E1B genes may be linked by an IRES between the E1A and E1B genes. In the construction of this virus, the endogenous E1B promoter elements are removed and replaced with the IRES element. Therefore both E1A and E1B expression are under the control of the inducer responsive promoter element. As an IRES alternative, the 2A peptide sequence derived foot and mouth disease virus (FMDV) could be used in place of the IRES sequence (as described in Furler S et al., Gene Ther. 2001 June;8(11):864-73) to provide efficient bicistronic expression of both E1A and a transgene.

In this way, various genetic capabilities may be introduced into target cells, particularly cancer cells. Alternatively, the vector may comprise a heterologous transgene encoding a therapeutic gene product under the control of a constitutive or inducible promoter. Numerous examples of constitutive and inducible promoters are known in the art and routinely employed in transgene expression in the context of viral or non-viral vectors. In this way, various genetic capabilities may be introduced into target cells. For example, in certain instances, it may be desirable to enhance the degree therapeutic efficacy by enhancing the rate of cytotoxic activity. This could be accomplished by coupling the cancer cell-specific TRE activity with expression of, one or more metabolic enzymes such as HSV-tk, nitroreductase, cytochrome P450 or cytosine deaminase (CD) which render cells capable of metabolizing 5-fluorocytosine (5-FC) to the chemotherapeutic agent 5-fluorouracil (5-FU), carboxylesterase (CA), deoxycytidine kinase (dCK), purine nucleoside phosphorylase (PNP), thymidine phosphorylase (TP), thymidine kinase (TK) or xanthine-guanine phosphoribosyl transferase (XGPRT). This type of transgene may also be used to confer a bystander effect.

Any gene or coding sequence of therapeutic relevance can be used in the practice of the invention. For example, genes encoding immunogenic polypeptides, toxins, immunotoxins and cytokines are useful in the practice of the invention. Additional transgenes that may be introduced into a vector of the invention include a factor capable of initiating apoptosis, antisense or ribozymes, which among other capabilities may be directed to mRNAs encoding proteins essential for proliferation, such as structural proteins, transcription factors, polymerases, etc., viral or other pathogenic proteins, where the pathogen proliferates intracellularly, cytotoxic proteins, e.g., the chains of diphtheria, ricin, abrin, etc., genes that encode an engineered cytoplasmic variant of a nuclease (e.g., RNase A) or protease (e.g., trypsin, papain, proteinase K, carboxypeptidase, etc.), chemokines, such as MCP3 alpha or MIP-1, pore-forming proteins derived from viruses, bacteria, or mammalian cells, fusogenic genes, chemotherapy sensitizing genes and radiation sensitizing genes.

Other genes of interest include cytokines, antigens, transmembrane proteins, and the like, such as IL-1, IL-2, IL-4, IL-5, IL-6, IL-10, IL-12, IL-18 or flt3, GM-CSF, G-CSF, M-CSF, IFN-α, -β, -γ, TNF-α, -β, TGF-α, -β, NGF, MDA-7 (Melanoma differentiation associated gene-7, mda-7/interleukin-24), and the like. Further examples include, proapoptotic genes such as Fas, Bax, Caspase, TRAIL, Fas ligands, nitric oxide synthase (NOS) and the like; fusion genes which can lead to cell fusion or facilitate cell fusion such as V22, VSV and the like; tumor suppressor gene such as p53, RB, p16, p17, W9 and the like; genes associated with the cell cycle and genes which encode anti-angiogenic proteins such as endostatin, angiostatin and the like.

Other opportunities for specific genetic modification include T cells, such as tumor infiltrating lymphocytes (TILs), where the TILs may be modified to enhance expansion, enhance cytotoxicity, reduce response to proliferation inhibitors, enhance expression of lymphokines, etc. One may also wish to enhance target cell vulnerability by providing for expression of specific surface membrane proteins, e.g., B7, SV40 T antigen mutants, etc.

Additional genes include the following: proteins that stimulate interactions with immune cells such as B7, CD28, MHC class I, MHC class II, TAPs, tumor-associated antigens such as immunogenic sequences from MART-1, gp 100(pmel-17), tyrosinase, tyrosinase-related protein 1, tyrosinase-related protein 2, melanocyte-stimulating hormone receptor, MAGE1, MAGE2, MAGE3, MAGE12, BAGE, GAGE, NY-ESO-1, β-catenin, MUM-1, CDK-4, caspase 8, KIA 0205, HLA-A2R1701, α-fetoprotein, telomerase catalytic protein, G-250, MUC-1, carcinoembryonic protein, p53, Her2/neu, triosephosphate isomerase, CDC-27, LDLR-FUT, telomerase reverse transcriptase, PSMA, cDNAs of antibodies that block inhibitory signals (CTLA4 blockade), chemokines (MIP1α, MIP3α, CCR7 ligand, and calreticulin), anti-angiogenic genes include, but are not limited to, genes that encode METH-I, METH-2, TrpRS fragments, proliferin-related protein, prolactin fragment, PEDF, vasostatin, various fragments of extracellular matrix proteins and growth factor/cytokine inhibitors, various fragments of extracellular matrix proteins which include, but are not limited to, angiostatin, endostatin, kininostatin, fibrinogen-E fragment, thrombospondin, tumstatin, canstatin, restin, growth factor/cytokine inhibitors which include, but are not limited to, VEGF/VEGFR antagonist, sFlt-1, sFlk, sNRP1, angiopoietin/tie antagonist, sTie-2, chemokines (IP-10, PF-4, Gro-beta, IFN-gamma (Mig), IFNα, FGF/FGFR antagonist (sFGFR), Ephrin/Eph antagonist (sEphB4 and sephrinB2), PDGF, TGFβ and IGF-1. Genes suitable for use in the practice of the invention can encode enzymes (such as, for example, urease, renin, thrombin, metalloproteases, nitric oxide synthase, superoxide dismutase, catalase and others known to those of skill in the art), enzyme inhibitors (such as, for example, alpha1-antitrypsin, antithrombin III, cellular or viral protease inhibitors, plasminogen activator inhibitor-1, tissue inhibitor of metalloproteases, etc.), the cystic fibrosis transmembrane conductance regulator (CFTR) protein, insulin, dystrophin, or a Major Histocompatibility Complex (MHC) antigen of class I or II. Also useful are genes encoding polypeptides that can modulate/regulate expression of corresponding genes, polypeptides capable of inhibiting a bacterial, parasitic or viral infection or its development (for example, antigenic polypeptides, antigenic epitopes, and transdominant protein variants inhibiting the action of a native protein by competition), apoptosis inducers or inhibitors (for example, Bax, Bc12, Bc1X and others known to those of skill in the art), cytostatic agents (e.g., p21, p16, Rb, etc.), apolipoproteins (e.g., ApoAI, ApoAIV, ApoE, etc.), oxygen radical scavengers, polypeptides having an anti-tumor effect, antibodies, toxins, immunotoxins, markers (e.g., beta-galactosidase, luciferase, etc.) or any other genes of interest that are recognized in the art as being useful for treatment or prevention of a clinical condition. Further therapeutic genes include a polypeptide which inhibits cellular division or signal transduction, a tumor suppressor gene (such as, for example, p53, Rb, p73), a polypeptide which activates the host immune system, a tumor-associated antigen (e.g., MUC-1, BRCA-1, an HPV early or late antigen such as E6, E7, L1, L2, etc), optionally in combination with a cytokine gene.

The invention further comprises combinations of two or more transgenes with synergistic, complementary and/or nonoverlapping toxicities and methods of action. The resulting adenovirus would retain the viral oncolytic functions and would, for example, additionally have the ability to induce immune and anti-angiogenic responses, etc.

In the vectors of the invention, a transgene/therapeutic gene or coding sequence thereof is under the control of a suitable promoter. Suitable promoters that may be employed include, but are not limited to, adenoviral promoters, such as the adenoviral major late promoter and/or the E3 promoter; the cytomegalovirus (CMV) promoter; the Rous Sarcoma Virus (RSV) promoter; inducible promoters, such as the MMT promoter, the metallothionein promoter; heat shock promoters; the albumin promoter; the ApoAI promoter; and a tissue-specific TRE such as found in the scientific literature, some examples of which a re described herein.

In one embodiment of the invention, the Hela-S3 cells are further modified to express a protein that binds a ligand on the adenovirus and enhances viral transduction of the cell. For example, if the adenovirus is targeted to a specific cell receptor, the Hela-S3 cell may be modified to express or up-regulate expression of the cell receptor to enhance viral infection. Adenoviruses are made by transferring vectors into packaging cells by techniques known to those skilled in the art. Packaging cells typically complement any functions deleted from the wildtype adenovirus genome. The production of such particles requires that the vector be replicated and that those proteins necessary for assembling an infectious virus be produced.

The packaging cells are cultured under conditions that permit the production of the desired viral particle. The particles are recovered by standard techniques.

In an effort to find an improved platform to produce oncolytic adenoviruses, candidate cell lines are initially screened as adherent lines and those that demonstrate ideal production on a particle/cell basis are selected for adaptation experiments. PER.C6 typically serves as the internal control for cell screening experiments, as it historically produces 25,000-50,000 viral particles/cell (vp/cell). Cell lines that produce equal or more vp/cell than PER.C6 or exhibit other desirable characteristics are selected for adaptation studies. An object is to transition the chosen cell lines from adherent to suspension cultures in serum-free culture media, because suspension cell lines are easier to scale up than adherent lines, making them more advantageous for a production process.

Viral particle production is one factor in establishing an adequate production platform. However, for a cell line to be considered fully adapted to suspension, serum-free conditions, criteria such as the following are also considered. Upon being introduced to serum-free media, cells preferably stay detached from the culture surface and begin growing in small grape-like clusters or as individual cells. Growth and viability of the cultures may be negatively affected when first introduced to serum-free media, but successful adaptation is characterized by steadily increasing culture viabilities and decreasing doubling times. When a cell line demonstrates viabilities of 90% or more and doubling times of 30 hours or less on a consistent basis, the cell line is taken to the next step of establishing cryopreservation conditions.

Cryopreservation is successful if, after being frozen, the cells thaw, scale up, and grow exhibiting 90% or greater viabilities and 30 hours or less doubling times. When these criteria are met, the cell line is considered to be adapted. From there, a working cell bank of the newly adapted suspension cell line is created to support future experiments.

Before a newly adapted cell line is transferred to large scale (bioreactor) studies, the production of the suspension cell on a small scale level is evaluated. Two issues are addressed. First, a determination whether vector production is cell passage dependent. To accomplish this, multiple production runs are carried out as the age of the cell culture increases. With each experiment, the cells are infected with a sample of the vector originating from a stock source. Secondly, a determination whether the virus can be sequentially passaged through the chosen cell line. This is done by performing an initial production experiment, harvesting and using that product to infect the cells on second, third, and fourth rounds etc. Several rounds of experiments are performed as necessary to determine the amount of virus produced and the biological activity of the virus at each round.

As shown below in the examples, HeLa-S3 has been successfully adapted as suspension cells in serum-free media and is effective to produce oncolytic adenoviruses. Production experiments described herein show that the HeLa-S3 cell line is an optimal platform for oncolytic adenovirus production.

### EXAMPLES

The present invention is described by reference to the following Examples, which are offered by way of illustration and are not intended to limit the invention in any manner. Standard techniques well known in the art or the techniques specifically described below are utilized.

### Example 1

### Cell Culture

FBS used to supplement media for adherent cell lines was not heat inactivated. PER.C6 adherent cells were initially grown in DMEM (Gibco)+10% FBS+10 mM MgCl₂. HeLa-S3 adherent cells were initially grown in DMEM supplemented with 10% FBS.

### Example 2

### Thawing Adherent Cell Lines

Cells are retrieved from a liquid nitrogen freezer and immediately placed on dry ice. Cells are thawed rapidly at 37° C. until just thawed (only a few minutes). The vial is taken to a biosafety cabinet (hood). The vial is inverted 2-3 times to mix cells. A 2 ml serological pipette is used to transfer the cells to a 15 ml conical centrifuge tube. 9 ml of appropriate serum containing media is added to the 1 ml cell suspension for a total of 10 ml. The 15 ml conical is briefly vortexed (medium speed) to make sure cells are evenly dispersed. A 2 ml serological pipette is used to transfer 0.2-0.3 ml (200-30011) to a 1.5 ml eppendorf tube to be used for cell counting. After the sample is taken, and before the cells are counted, the remaining cells are centrifuged at 1000 rpm for 5 minutes. The cells are counted during centrifugation.

The cells are removed from the centrifuge and the DMSO-containing media is aspirated off using a serological aspirating pipette. The cells are resuspended in 10 ml fresh media. From the cell count results, the number of cells/ml in the 10 ml is calculated. Cells/ml is multiplied by 10 ml to determine total cells. Cells are seeded as cells/cm² at a density of 3-4 cells/cm². The size of the vessel suitable to achieve the desired cell density is calculated based on the total cells, which may be accomplished by dividing total cells by the surface area of the flask. The T-Flask is placed as a static culture (no rocking or agitation) in an incubator at 37° C., 5% CO₂, and 84% humidity.

### Example 3

### Cryopreservation of Adherent and Suspension Cell Lines

The following is a general method for cryopreserving adherent and suspension cells. Adherent Cell Lines: Adherent cells scheduled to be frozen are scaled up in T-175 flasks. The cells are typsinized and counted. The volume of quenched cell suspension needed to make a stock cell suspension at 2e6 cells/ml is determined. Cells are centrifuged out of quenched media for five minutes at 1000 rpm and resuspended in conditioned media at a volume resulting in 2e6 cells/ml. A stock solution of freeze media consisting of fresh media supplemented with 20% DMSO is made. 2e6 cells/ml suspension is diluted with freeze media in a ratio of 1:1, resulting in a cell suspension with a density of 1e6 cells/ml and 10% DMSO in conditioned/fresh media. 1 ml/cryovial is aliquoted into the desired number of cryovials.

Suspension Cell Lines: Suspension cells scheduled to be frozen are scaled up in roller bottles. The bottle is swirled to achieve an adequate dispersion of cells throughout the media and 1-1.5 ml is aspirated off to use for cell counting. The volume of cell suspension needed to attain a stock 2e6 cells/ml suspension is determined. That volume is centrifuged for five minutes at 1000 rpm and resuspended in a volume of conditioned media, resulting in a 2e6 cells/ml stock. Freeze media is made consisting of fresh media supplemented with 15% DMSO and 5% or 10% sucrose (if needed). Sucrose is made up as a 20% solution in the base media and then added to the freeze media. The 2e6 cells/ml stock is diluted with freeze media in a 1:1 ratio to attain a cell suspension at 1e6 cells/ml, 7.5% DMSO in conditioned/fresh media. 1 ml/cryovial is aliquoted into the desired number of cryovials.

### Example 4

### Harvesting and Preparing Whole, Supernatant, and Pellet Samples

The following applies to harvesting samples for virus production experiments using cells that have already been adapted to suspension culture in serum-free media. From any given culture, the flask or roller bottle is swirled to achieve a homogenous suspension. To attain a whole sample, a serological pipette is used to aspirate off 5 ml and place it into a 15 ml conical tube. This tube is placed in a −80° C. freezer. To attain supernatant and pellet samples, the culture is swirled again. 10 ml is aspirated off and placed into a 15 ml conical tube. This tube is centrifuged at 3000 rpm for 5 minutes. 2 ml supernatant is aspirated off and placed into a 2 ml cryovial. The pellet is resuspended in 1 or 2 ml PBS or conditioned media to achieve a 10× or 5× pellet, respectively. The supernatant and pellet samples are placed in a −80° C. freezer. The samples are removed from the −80° C. freezer and placed on dry ice to prepare them for analysis by Hexon and HPLC. A series of freeze/thaws is performed on the whole and pellet samples to lyse the cells and release the virus into the media. After 3 rounds of freeze/thaw, samples are centrifuged for 5 minutes at 3000 rpm. The cleared viral lysate (CVL) from each sample is drawn off and placed in 2 ml cryovials. Samples are then ready to be analyzed.

### Example 5

### Quantification of Virus Production

HPLC—The adenovirus particles are bound to a Pharmacia Biotech Resource Q anion exchange column and separated from other components by an NaCl gradient elution. The elution profile is obtained through on-line measurement of absorbance at 260 nm. The 260 nm signal is used for routine quantitation of the adenovirus particles. The peak area is used to calculate the final viral concentration using a linear standard curve generated on the same HPLC system.

Flow Cytometry—A biological assay based on immunodetection of hexon in infected cells is used to determine the virus concentration in various samples. Virus-containing samples are serially diluted and inoculated onto dexamethasone-induced AE1-2a cells (Gorziglia et al., J. Virol. 70: 4173-4178 (1996)). 24 hours post-inoculation, the AE1-2A cells are harvested and fixed overnight. The following day the cells are permeabilized, stained with anti-hexon antibody conjugated to FITC, then acquired by flow cytometry on a FACS Calibur. The concentration of virus in the inoculate is calculated by determining the dilution at which 50% of the infected cell population expresses hexon (EC₅₀) and comparing this with the EC₅₀ of an internal vector standard of known concentration as determined by standard OD₂₆₀ reading. Spectrophotometric Analysis—Virus particle concentrations may also be determined by spectrophotometric analysis as described by Mittereder et al. (J. Virol. 70: 7498-7509 (1996)).

### Example 6

### Adaptation to Serum-Free Media

Direct adaptation typically involves two methods. First, the adherent cell line is seeded in a T-Flask with the base/serum containing media. At 24 hrs post-seeding, the media is aspirated off, cells are washed with PBS and introduced to 100% serum-free media. Secondly, when thawing a vial of adherent cells, after aspirating off the DMSO/serum containing media, the cell pellet is resuspended in 100% serum-free media and a T-flask is seeded.

Weaned adaptation involves seeding the cells in the base/serum containing media. Then, 24 hours post-seeding, the culture media is aspirated and a 50:50 mix of serum media:serum-free media is added to the culture. The cells are then allowed to grow in that media. Upon cell passaging the cells are again seeded in the 50:50 mix and then at 24 hours post-seeding, the culture is introduced to a 25:75 mix of serum:serum-free media. The cells are handled in this fashion until they are in 100% serum-free media.

Other adaptation methods include seeding cells in shaker flasks and on T-flask rockers to prevent cell clumping/settling, using dextran sulfate to also prevent cell clumping, using serum albumin replacements, and going straight from T-flasks to roller bottles.

HeLa-S3 was adapt to both EX-CELL™ 293 (JRH Biosciences, Lenexa, Kans., USA) and CD293 (GIBCO™ Invitrogen Corporation, Carlsbad, Calif., USA) serum-free media. Cells were initially grown in D10 and then seeded into shaker flasks in 100% EX-CELL™ 293 or CD293 media. Both were eventually transitioned from shaker flasks to roller bottles. At that point, cells were frozen in two different cryopreservation formulas per media. One formula is 100% EX-CELL™ 293 (50% conditioned and 42.5% fresh) with 7.5% DMSO. The second Formula is 100% EX-CELL™ 293 or CD293 (50% conditioned and 37.5% fresh) with 7.5% DMSO and 5% sucrose. Five vials for each condition are frozen.

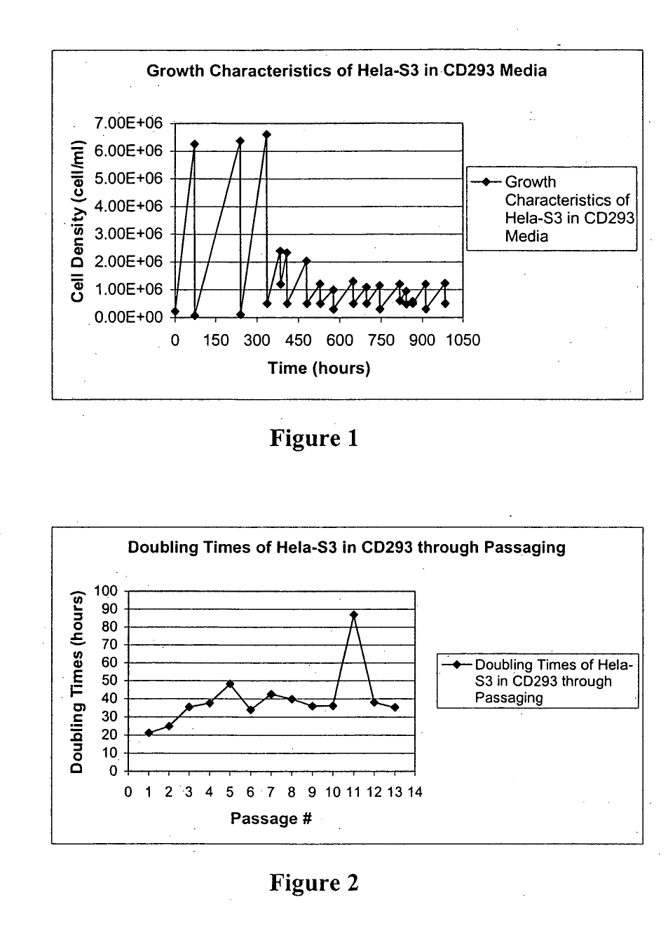

Tables 1 and 2 (and FIGS. 1 and 2) represent growth characteristics and doubling times in CD293 media. Table 1 (and FIG. 1) shows seeding/harvest densities for a given portion of the cell culture's life span. The first 300-350 hours are during adaptation; therefore, during this stage cells are still adherent and grow to higher densities compared to when the cells go into suspension approximately 400 hours post-thaw. Harvest densities after being adapted generally reach just over 1e6 cells/ml. Table 2 (and FIG. 2) shows doubling times after the cells go into suspension; it does not include the adaptation stage. This data correlates roughly to hours 400 and on in Table 1 (FIG. 1). Doubling times steadily increased in this culture, stabilizing at between 35 and 40 hours.

TABLE 1
HeLa-S3 Cell Line History and Adaptation to CD293
Serum-Free Media

| Time (hours) | Cell Density (cells/ml) |
|---|---|
| 0 | 2.20E+05 |
| 72 | 6.25E+06 |
| 73 | 7.00E+04 |
| 240 | 6.37E+06 |
| 241 | 1.05E+05 |
| 336 | 6.60E+06 |
| 337 | 5.00E+05 |
| 384 | 2.40E+06 |
| 385 | 1.20E+06 |
| 408 | 2.34E+06 |
| 409 | 5.00E+05 |
| 480 | 2.04E+06 |
| 481 | 5.00E+05 |
| 528 | 1.21E+06 |
| 529 | 5.00E+05 |
| 576 | 9.96E+05 |
| 577 | 3.00E+05 |
| 648 | 1.30E+06 |
| 649 | 5.00E+05 |
| 696 | 1.09E+06 |
| 697 | 5.00E+05 |
| 744 | 1.15E+06 |
| 745 | 3.00E+05 |
| 816 | 1.20E+06 |
| 817 | 6.00E+05 |
| 840 | 9.50E+05 |
| 841 | 4.75E+05 |
| 864 | 5.75E+05 |
| 865 | 5.00E+05 |
| 912 | 1.20E+06 |
| 913 | 3.00E+05 |
| 984 | 1.23E+06 |
| 985 | 5.00E+05 |

TABLE 2
HeLa-S3 Cell Line History and Adaptation to CD293 Serum-Free
Media
Doubling Times and Passage #'s

| Passage # | DT (hrs) |
|---|---|
| 1 | 21.2 |
| 2 | 24.9 |
| 3 | 35.5 |
| 4 | 37.6 |
| 5 | 48.2 |
| 6 | 34 |
| 7 | 42.6 |
| 8 | 39.9 |
| 9 | 36 |
| 10 | 36.2 |
| 11 | 87 |
| 12 | 38 |
| 13 | 35.3 |

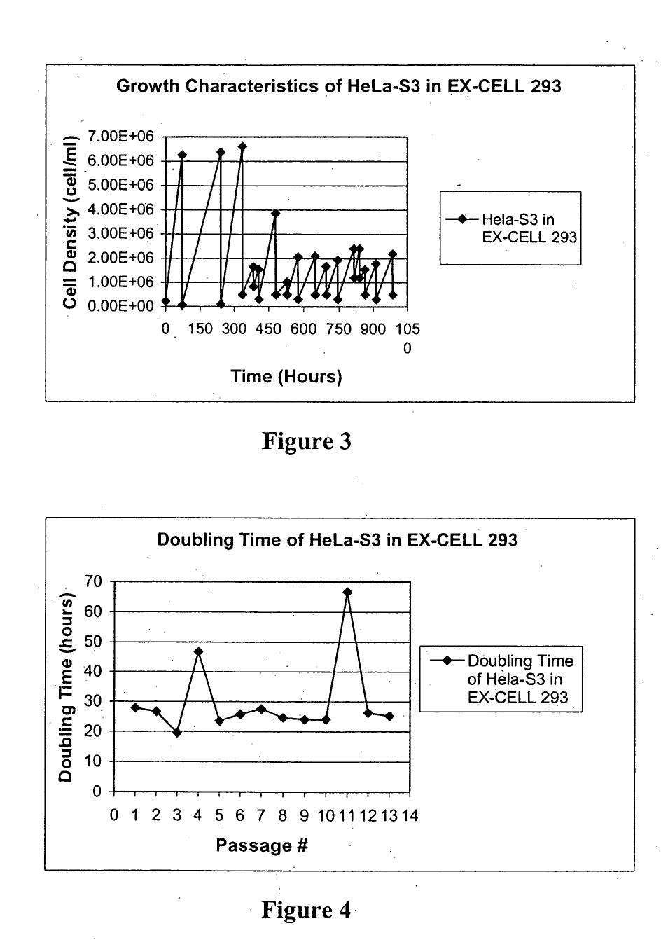

Tables 3 and 4 (and FIGS. 3 and 4) represent growth characteristics and doubling times in EX-CELL™ 293 media. Table 3 (and FIG. 3) shows seeding/harvest densities for a given portion of the cell culture's life span. The first 300-350 hours are during adaptation; therefore, daring this stage cells are still adherent and grow to higher densities compared to when the cells go into suspension approximately 400 hours post-thaw. Once the cells were put into suspension in 100% EX-CELL™ 293 media (approximately hour 400 and on), the cells typically reached harvest densities between 1.7e6 and just over 2e6 cells/ml. Table 4 (and FIG. 4) shows doubling times after the cells were put into suspension. This data correlates roughly to hours 400 and beyond in Table 3 (FIG. 3). Doubling times generally remain in a range of 25 to 30 hours.

TABLE 3
HeLa-S3 Cell Line History and Adaptation to EX-CELL ™ 293
Serum-Free Media

| Time (hrs) | Cell Density (cells/ml) |
|---|---|
| 0 | 2.20E+05 |
| 72 | 6.25E+06 |
| 73 | 7.00E+04 |
| 240 | 6.37E+06 |
| 241 | 1.05E+05 |
| 336 | 6.60E+06 |
| 337 | 5.00E+05 |
| 384 | 1.65E+06 |
| 385 | 8.25E+05 |
| 408 | 1.54E+06 |
| 409 | 3.00E+05 |
| 480 | 3.86E+06 |
| 481 | 5.00E+05 |
| 528 | 1.02E+06 |
| 529 | 5.00E+05 |
| 576 | 2.06E+06 |
| 577 | 3.00E+05 |
| 648 | 2.09E+06 |
| 649 | 5.00E+05 |
| 696 | 1.67E+06 |
| 697 | 5.00E+05 |
| 744 | 1.93E+06 |
| 745 | 3.00E+05 |
| 816 | 2.40E+06 |
| 817 | 1.20E+06 |
| 840 | 2.40E+06 |
| 841 | 1.20E+06 |
| 864 | 1.54E+06 |
| 865 | 5.00E+05 |
| 912 | 1.78E+06 |
| 913 | 3.00E+05 |
| 984 | 2.18E+06 |
| 985 | 5.00E+05 |

TABLE 4
HeLa-S3 Cell Line History and Adaptation to EX-CELL ™ 293
Serum-Free Media
Doubling Times and Passage #'s

| Passage # | DT (hrs) |
|---|---|
| 1 | 27.8 |
| 2 | 26.6 |
| 3 | 19.5 |
| 4 | 46.6 |
| 5 | 23.5 |
| 6 | 25.7 |
| 7 | 27.5 |
| 8 | 24.6 |
| 9 | 24 |
| 10 | 24 |
| 11 | 66.6 |
| 12 | 26.2 |
| 13 | 25.1 |

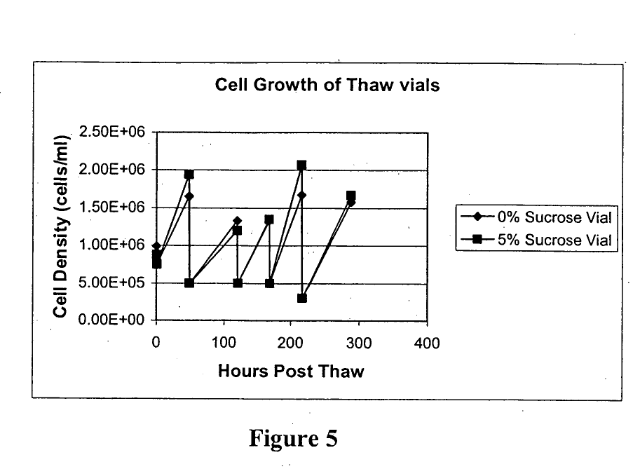

The thaw testing history of HeLa-S3 in EX-CELL™ 293 serum-free media is represented in Table 5 (and FIG. 5). Table 5 (and FIG. 5) shows growth characteristics for HeLa-S3 suspension line adapted in EX-CELL™ 293 media, but frozen with and without 5% sucrose. The results are almost identical. Therefore, a working cell bank may be frozen with or without sucrose.

TABLE 5
HeLa-S3 in EX-CELL ™ 293: Thaw Testing History
Data for Cell Growth Chart

| | |
|---|---|
| 0% Sucrose Vial | 5% Sucrose Vial |

| hours | density | hours | density |
|---|---|---|---|
| 0 | 9.90E+05 | 0 | 8.76E+05 |
| 1 | 7.50E+05 | 1 | 7.50E+05 |
| 48 | 1.65E+06 | 48 | 1.94E+06 |
| 49 | 5.00E+05 | 49 | 5.00E+05 |
| 120 | 1.33E+06 | 120 | 1.20E+06 |
| 121 | 5.00E+05 | 121 | 5.00E+05 |
| 168 | 1.33E+06 | 168 | 1.35E+06 |
| 169 | 5.00E+05 | 169 | 5.00E+05 |
| 216 | 1.67E+06 | 216 | 2.07E+06 |
| 217 | 3.00E+05 | 217 | 3.00E+05 |
| 288 | 1.58E+06 | 288 | 1.67E+06 |

In conclusion, as shown in Tables 1-5 and FIGS. 1-5, the HeLa-S3 cells demonstrate a shorter doubling time in EX-CELL™ 293 media. Over the course of the culture, the doubling times in EX-CELL™ 293 media are more stable and remain in a range of 25 to 30 hours, and harvest densities consistently reach between 1.7 and 2e6 cells/ml. In contrast, doubling times in CD293 slowly increase during the life of the culture, ranging from 36 to 40 hours, and harvest densities are lower.

### Example 7

### Production of Oncolytic Adenovirus in HeLa-S3 Cells

The series of experiments described below involve HeLa-S3 cells and oncolytic adenovirus vector Ar17E2fFTrtex (PCT/US02/05300 (WO 02/067861)) compared with oncolytic adenovirus vector Ar6pAE2fE3F (PCT/US02/05280 (WO 02/068627); PCT/US02/05300 (WO 02/067861)).

Step One—Thawing and Scaling up of Cells: The HeLa-S3 cells are thawed as adherent. Thirty seven days post-thaw, the cells are adapted as described above and ready to be seeded for the first production experiment. Both the EX-CELL™ 293 and CD293 lines are used in the early rounds of experimentation.

Step Two—General Setup of Experiments: The number of T-75 flasks needed for each experiment is dependent on the number of viruses being screened. On the day of infection for any given experiment, cells are counted and then spun out of conditioned EX-CELL™ 293 or CD293 media. During centrifugation, a calculation based on the cell count is done to determine how many cells are needed to seed a T-75 flask in 15 ml at a density of 1e6 cells/ml. That cell number is actually resuspended in 14 ml of fresh media. After seeding, 1 ml of viral inoculant is added to the bottle to achieve an infection concentration of 2e8 vp/ml in 15 ml total. For purified viral preps, titers are measured by an optical density reading (Mittereder et al., J. Virol. 70: 7498-7509 (1996)). CVL titers are determined by HPLC as described above. At 24 hours post infection, cultures are fed 1:1 with fresh EX-CELL™ 293 media. At 48 hours post infection, samples are harvested. Samples are generally whole samples, and are sometimes supernatant samples and pellet samples. Analysis of samples is performed by Hexon FACS and by HPLC.

37 Days Post-Thaw: Vialed aliquots of Ar6pAE2fE3F and Ar17E2fFTrtex were used to infect the HeLa-S3 cells. The experiment was set up as detailed in Step Two above. For this round there were two harvest points, one at 48 hours and 72 hours.

53 Days Post-Thaw: Cells were infected with a fresh stock of Ar6pAE2fE3F and Ar17E2fFTrtex. Whole samples and pellet samples were harvested around 70 hours post infection. Products are labeled “HL1” and were not purified over cesium chloride.

60 Days Post-Thaw: Cells were infected with stock Ar6pAE2fE3F, CVL Ar6pAE2fE3F HL1, Ar17E2fFTrtex, and CVL Ar17E2fFTrtex HL1. Whole samples and pellet samples were harvested around 70 hours post-infection. Products from this experiment are labeled “HL1HL2” and were not purified over cesium chloride.

74 Days Post-Thaw: Cells were infected with stock Ar6pAE2fE3F, CVL Ar6pAE2fE3F HL1HL2, Ar17E2fFTrtex, and CVL Ar17E2fFTrtex HL1HL2. Whole samples and pellet samples were harvested at 70 hours post-infection. Products from this experiment are labeled “HL1HL2HL3” and were not purified over cesium chloride.

88 Days Post-Thaw: Cells adapted to EX-CELL™ 293 were infected with stock Ar6pAE2fE3F, CVL Ar6pAE2fE3F HL1HL2HL3, Ar17E2fFTrtex, and CVL Ar17E2fFTrtex HL1HL2HL3. Whole samples and pellet samples were harvested 70 hours post-infection. Products from this experiment are labeled “HL1HL2HL3HL4” and were not purified over cesium chloride.

116 Days Post-Thaw: Cells were infected with stock Ar6pAE2fE3F, CVL Ar6pAE2fE3F HL1HL2HL3HL4, Ar17E2fFTrtex, and CVL Ar17E2fFTrtex HL1HL2HL3HL4 (to produce Ar6pAE2fE3F HL1→HL5 and Ar17E2fFTrtex HL1→HL5). Whole samples and pellet samples were harvested at 70 hours post-infection.

All samples from very round of experimentation were analyzed by Hexon (FACS) and HPLC. Table 6 shows data points analyzed by HPLC for HeLa-S3 for six rounds of an exemplary study. The first data points show that viral production started close to 1e11 vp/ml. although there is a slight downward trend with the HPLC data, the reduction is less than 2-fold from the initial data set. The last data sets show viral particle production trending back up. This trend is the same for all three lines tested, Ar6pAE2fE3F, Ar17E2fFTrtex, and the sequentially passaged Ar17E2fFTrtex H-Line.

Table 7 shows the number of biologically active viral particles (vp/ml). Again, all three lines trend the same with increases on the third and sixth data points. Comparing the Ar6pAE2fE3F line in Table 7 to the Ar6pAE2fE3F line in Table 6, the total particles produced appear to be mostly biologically active. Comparing the data for Ar17E2fFTrtex, and the sequentially passaged Ar17E2fFTrtex H-Line in Table 7 with the data in Table 6 shows the biologically active particles to be less than the total particles, but only by a one log difference.

TABLE 6
Effect of Cell Age on Virus Production
HeLa-S3, EX-CELL ™ 293, HPLC Data, Whole Sample

| | Virus (vp/ml) | | |
|---|---|---|---|
| Days post-thaw | Ar6pAE2fE3F | Ar17E2fFTrtex | Ar17E2fFTrtex H-Line |
| 37 | 8.15E+10 | 8.70E+10 | N/A |
| 53 | 8.47E+10 | 7.47E+10 | 7.47E+10 |
| 60 | 7.51E+10 | 7.44E+10 | 7.52E+10 |
| 74 | 6.28E+10 | 5.60E+10 | 5.38E+10 |
| 88 | 4.41E+10 | 4.73E+10 | 4.14E+10 |
| 116 | 5.49E+10 | 6.05E+10 | 5.21E+10 |

TABLE 7
Effect of Cell Age on Biological Activity of Virus Produced
HeLa-S3, EX-CELL ™ 293, Hexon FACS Data, Whole Sample

| | Virus (vp/ml) | | |
|---|---|---|---|
| Days post-thaw | Ar6pAE2fE3F | Ar17E2fFTrtex | Ar17E2fFTrtex H-Line |
| 37 | 1.25E+11 | 2.24E+10 | N/A |
| 53 | 5.28E+10 | 9.17E+09 | 9.17E+09 |
| 60 | 8.86E+10 | 2.13E+10 | 1.43E+10 |
| 74 | 3.90E+10 | 8.89E+09 | 6.04E+09 |
| 88 | 2.59E+10 | 3.83E+09 | 2.64E+09 |
| 116 | 3.40E+10 | 1.03E+10 | 1.14E+10 |

This data shows that HeLa-S3 cells can successfully grow oncolytic adenoviruses such as Ar6pAE2fE3F and Ar17E2fFTrtex, and that the viruses can be sequentially passaged on this cell line.

### Example 8

### Production of Oncolytic Adenovirus in HeLa-S3 Cells

Total process time from thaw of MCB harvest of the suspension HeLa-S3 cells is 28 days. In that time the culture is scaled to a working volume of 30 L and infected with MVB derived CG0070.

CG0070 is a selectively replicating adenovirus that comprises in sequential order a LITR, an adenovirus packaging signal, a SV40 pA, an E2f 1TRE operatively linked to the E1a coding sequence and a human GM-CSF coding sequence inserted in place of the E3 GP19 coding sequence.

Step One—Thawing and Scaling up of Cells: The HeLa-S3 cells were thawed as a suspension culture in Ex-Cell 293 media containing 6 mM L-glutamine (growth media). In 16 days, one vial of HeLa-S3 culture was scaled to 1.5 L in disposable shake. An example of HeLa-S3 scale-up in growth media is listed in Table 8.

Step Two—15 L Instrumented Spinner: The 1.5 L of culture generated from Step One and growth media were used to inoculate a 15 L instrumented spinner at a working volume of 4.5 L. The culture within the 15 L spinner maintains dissolved oxygen and temperature by use of a biocontroller. Temperature was maintained at 37 C and dissolved oxygen at 50% of air saturation. The culture was scaled-up in the 15 L vessel to a total volume of 10 L in 5 days, 21 days from thaw and was then transferred to a 30 L instrumented spinner. An example of HeLa -S3, 2× passage in the 15 L Instrumented spinner is listed in Table 9.

Step Three—30 L Instrumented Spinner: After 21 days from thaw, the 30 L vessel was inoculated with cells from the 15 L and fresh growth media to a volume of 30 L. The 30 L vessel maintains dissolved oxygen (50% of air saturation), temperature (37 C) and pH (7.2) by use of a biocontroller. The culture continues to grow in batch mode for 3 days from inoculation and 24 days from thaw. An example of HeLa-S3 30 L growth performance in the 30 L Instrumented spinner is listed in Table 10.

Step Four—Perfusion: After three days of growth, perfusion through a disposable hollow fiber cartridge was started. Perfusion is the process in which spent media in the vessel is replaced with fresh growth media without removing the cells within the vessel. The culture was perfused until harvest, 7 days from inoculation, 28 days from thaw.

Step Five—Infection: After 1 day of perfusion, 25 days from thaw, the 30 L culture was infected with CG0070 derived from the MVB at a concentration of 5e8vp/mL of culture fluid (1.5e13 total viral particles).

Step six—Harvest: 7 days post inoculation, the culture was harvested. A cocktail of Lysis detergent (Triton X-100), magnesium chloride, and benzonase is added to the culture 30 minutes prior to harvesting. The lysed culture was then transferred to sterile disposable bags and purified.

Samples are taken throughout the culture operation for density, viability (Trypan Blue Exclusion Method), metabolite and titer evaluation. Titer was determined by HPLC anion exchange method. Four 30 L productions runs have been performed under the conditions listed above. Data from those runs is provided in Table 11. The 30 L process has been shown to be a robust process yielding consistent titers on average of 5.4e11+/−1.1e11vp/mL and very consistent specific productivities of 8.3e4+/−5.1e3vp/cell for the 4 runs.

TABLE 8
Thaw to 15 L Spinner Scale-up

| Days | | Working | Viable |
|---|---|---|---|
| in | | volume | cells |
| culture | Vessel size | (mL) | (cells/mL) |
| 0 | T-75 | 10 | 7.3e5 |
| 3 | T-75 | 10 | 3.8e5 |
| 4 | T-75 | 10 | 4.4e5 |
| 5 | T-75 | 10 | 7.6e5 |
| 7 | T-75 | 10 | 2.0e6 |
| 7 | 250 mL SF | 40 | 2.0e5 |
| 10 | 250 mL SF | 40 | 1.2e6 |
| 10 | 500 mL SF | 125 | 3.5e5 |
| 11 | 500 mL SF | 125 | 8.0e5 |
| 12 | 500 mL SF | 125 | 1.5e6 |
| 12 | 500 mL SF | 125 | 3.5e5 |
| 14 | 500 mL SF | 125 | 1.7e6 |
| 14 | 1 L SF | 500 | 1.6e5 |
| 17 | 1 L SF | 500 | 1.4e6 |
| 17 | 2 L SF × 2 | 750 × 2 | 3.5e5 |
| 19 | 2 L SF × 2 | 750 × 2 | 9.7e5 |

TABLE 9
Two Passages in 15 L Instrumented Spinner

| | | | Volume in | Density |
|---|---|---|---|---|
| Day | Vessel | Passage in vessel | vessel | Cells/mL |
| 0 | 15 L Spinner | Psg 1 | 4.5 L | 4.0E+05 |
| 1 | 15 L Spinner | Psg 1 | 4.5 L | 9.6E+05 |
| 2 | 15 L Spinner | Psg 1 | 4.5 L | 1.6E+05 |
| 2 | 15 L Spinner | Psg 2 | 10 L | 2.0E+05 |
| 3 | 15 L Spinner | Psg 2 | 10 L | 4.0E+05 |
| 4 | 15 L Spinner | Psg 2 | 10 L | 6.9E+05 |
| 5 | 15 L Spinner | Psg 2 | 10 L | 1.4E+06 |

TABLE 10
30 L Instrumented Spinner (30 L working volume)

| Time | Density | Glucose | Lactate | Glutamine | Perfusion |
|---|---|---|---|---|---|
| (Days) | (Cells/mL) | (g/L) | (g/L) | (mM) | Setting L/day |
| 0.0 | 2.8E+05 | 5.36 | 2.1E−01 | 4.42 | N/A |
| 0.8 | 5.2E+05 | 5.27 | 4.7E−01 | 4.17 | N/A |
| 1.1 | 6.8E+05 | 5.06 | 5.7E−01 | 3.1 | N/A |
| 1.7 | 1.1E+06 | 4.56 | 9.3E−01 | 3.19 | N/A |
| 2.0 | 1.4E+06 | 4.35 | 1.1E+00 | 3.06 | N/A |
| 2.8 | 2.6E+06 | 2.94 | 2.0E+00 | 1.98 | N/A |
| 3.0 | 3.2E+06 | 3.01 | 2.0E+00 | 2.31 | 30 |
| 3.8 | 5.1E+06 | 2.68 | 2.2E+00 | 2.91 | 45 |
| 4.0 | 5.7E+06 | 2.33 | 2.4E+00 | 2.71 | 45 |
| 4.8 | 7.5E+06 | 0.81 | 3.4E+00 | 1.76 | 45 |
| 5.8 | 5.9E+06 | 0.48 | 4.3E+00 | 2.1 | 45 |
| 6.8 | 4.3E+06 | 1.03 | 4.2E+00 | 2.75 | 45 |

TABLE 11
Titer and Specific Productivity
(4 separate 30 L runs)

| | | Specific |
|---|---|---|
| | Titer | Prod |
| 30 L Run # | vp/mL | vp/cell |
| Run 1 | 5.3E+11 | 8.6E+04 |
| Run 2 | 7.0E+11 | 8.8E+04 |
| Run 3 | 4.5E+11 | 8.0E+04 |
| Run 4 | 4.7E+11 | 7.7E+04 |

### Example 9

### Summary of the Process for Purification of Ad from HeLa Cell Production

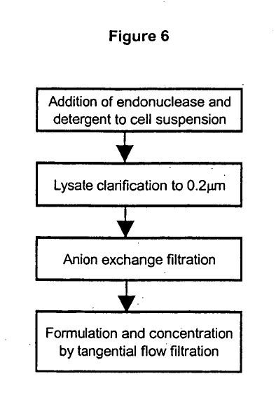

The purification process flow diagram is shown in FIG. 6. The harvested cell suspension is lysed using detergent, e.g. Triton X-100, in the presence of an endonuclease, e.g. Benzonase™, releasing the intracellular virus and yielding a crude lysate with a low burden of host cell, HPV and free adenoviral DNA. The lysis is preferably conducted at a conductivity of 10-50 mS, and a pH of 7.0-8.5. The crude lysate is clarified of debris greater than 0.2 mm by serial filtration. The clarified lysate is bound to a quaternary-amine-derivatized anion exchange filter (Q filter), preferably Pall Mustang Q, and the product eluted from the filter using a buffer of increased conductivity. See, e.g., WO 0307859. The preferred conductivities for loading and eluting the Q filter are 10-50 and 50-70 mS respectively, and the preferred pH of each step is 7.0-8.5. The Q filter step clears host cell and viral protein impurities, as well as trace DNA, production medium components and the endonuclease used during lysis. The product eluted from the Q filter is concentrated and formulated by tangential flow filtration (TFF), preferably having a nominal molecular weight cut-off of 750 kDa. The TFF step clears trace impurities as well as allows control of the final product concentration to titers as high as 4e12 vp/mL. The viral particle recovery for purification trials used to demonstrate the process is summarized in Table 12. The ability of the process to clear HPV DNA originating in the production culture is summarized in Table 13.

TABLE 12
Viral particle recovery over several lots

| | VP Step Recovery (by lot) | | | | |
|---|---|---|---|---|---|
| Step | 1671-030 | R2(1603-069) | R3/1603-072 | R3/1603-076 | R3/1603-081 |
| Crude | N/A | N/A | N/A | | |
| Clarification | 77% | 98% | 109% | | |
| MQ Eluate | 86% | 87% | 80% | 73% | 83% |
| TFF | 94% | 86% | 94% | 96% | 89% |
| Overall | 62% | 74% | 82% | 76% | 80% |

TABLE 13
HPV DNA clearance for several purified lots

| | 407 bp (7084-7630) | | |
|---|---|---|---|
| | HPV DNA | | |
| | [pg/4E11 Ad vp] | | |
| Lot | Crude | TFF | log clearance |
| PD3 | >12500 | <5 | >3.4 |
| PD4 | >12500 | <5 | >3.4 |
| 1671-030 | >12500 | <5 | >3.4 |
| R2/1603-051 | >12500 | <5 | >3.4 |
| R3/1603-072 | >12500 | <5 | >3.4 |
| R3/1603-076 | >12500 | <5 | >3.4 |
| R3/1603-081 | >12500 | <5 | >3.4 |

### Example 10

### Viral Burst Size in Hela-S3 Cells

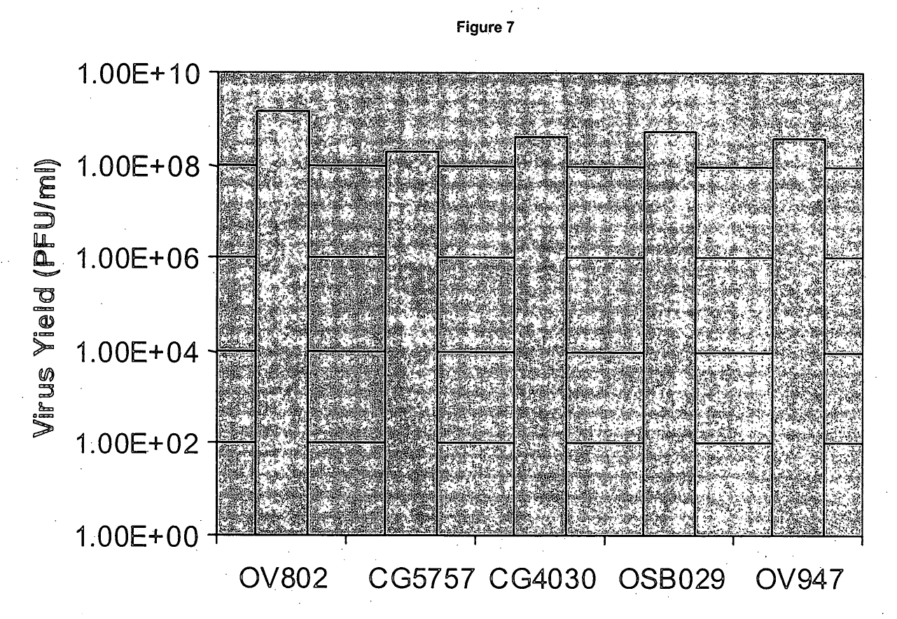

Virus burst size was determined by infecting 50 ml of Hela-S3 cells at 1e6/ml with different adenoviruses at 200 viral particles per cell. Cells were infected with the following adenoviruses: OV802 which is wildtype Adenovirus type 5; CG5757 has a human E2F TRE operatively linked to E1a and a hTERT TRE linked to E1b wherein the E1b region comprises a deletion in the 19K coding region; CG4030 comprises in sequential order a ITR, a SV40 pA, a human E2F TRE operatively linked to E1a and a hTERT TRE linked to E4, an adenoviral packaging signal and a ITR; OSB029 is similar to CG4030 except the adenovirus packaging signal is located in the wildtype position i.e. between the left ITR and the SV40 pA; OV947 is similar to CG5757 except that the E1b coding region contains the wildtype sequence and does not have a deletion in E1b 19K. After 96 hrs, infected cells were harvested and freeze/thawed for three times and then titrated on 293-E4 cells (Microbix Inc, Toronto, Ontario, Canada). The results are shown in FIG. 7. All five viruses produced greater than 10e8 PFUs (plaque forming units) from the Hela-S3 cells.

### Example 11

### Virus Growth Kinetics on Hela-S3 Cells

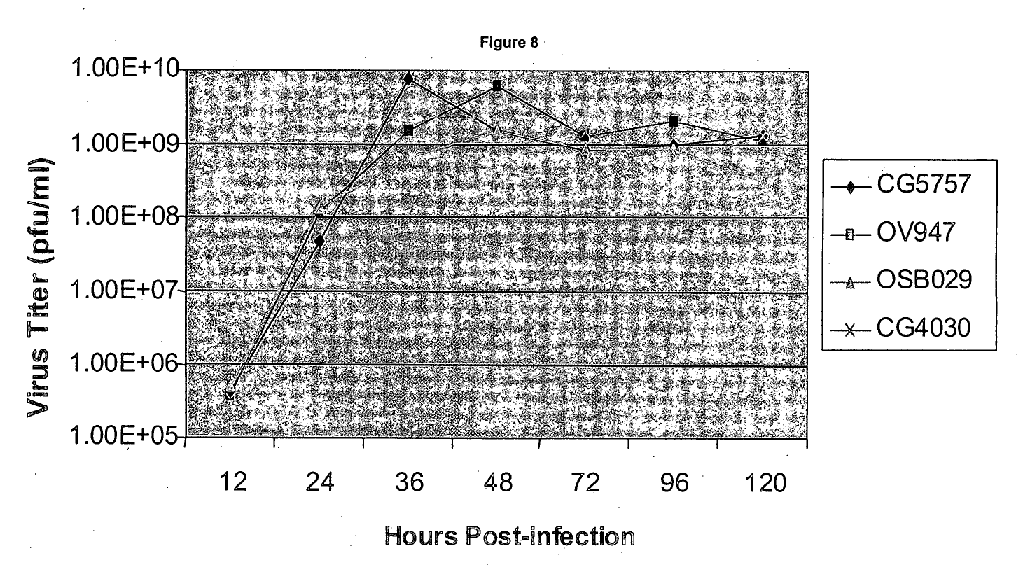

HeLa-S3 cells were infected at 2 pfu/cell in 200 ml culture at 1e6 cells/ml. 5 ml of infected cells were harvested at 12, 24, 36, 48, 72, 96 and 120 hours post infection (h.p.i.) and titrated on 293-E4 cells (Microbix Inc, Toronto, Ontario, Canada). The results are shown in FIG. 8. The CG5757 titer peaked at 36 h.p.i. and dropped slightly after 36 h.p.i. Other viruses had a titer peak at 48 h.p.i.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

1. A HeLa-S3 cell comprising a replication-competent adenovirus vector.

2. The HeLa-S3 cell of claim 1, wherein said replication-competent adenovirus vector is tumor or tissue-specific.

3. The HeLa-S3 cell of claim 1, wherein said tumor-specific replication-competent adenovirus vector comprises a mutation or deletion in the E1b gene, wherein the encoded E1b protein lacks the capacity to bind p53.

4. The HeLa-S3 cell of claim 1, wherein said tumor-specific replication-competent adenovirus vector comprises a mutation or deletion in the E1a gene, wherein the encoded E1a protein lacks the capacity to bind RB.

5. The HeLa-S3 cell of claim 1, wherein said vector comprises a heterologous transcriptional regulatory element (TRE) sequence operatively linked to the coding region of a gene that is essential for replication of said vector, wherein said TRE functions in said cell so that replication of the vector occurs in said cell.

6. The HeLa-S3 cell of claim 5, wherein said TRE comprises a promoter or enhancer.

7. The HeLa-S3 cell of claim 5, wherein said TRE is selected from the group consisting of an E2F-responsive TRE, a human telomerase reverse transcriptase (hTERT) TRE, an osteocalcin TRE, a carcinoembryonic antigen (CEA) TRE, a DF3 TRE, an α-fetoprotein TRE, an ErbB2 TRE, a surfactant TRE, a tyrosinase TRE, a PRL-3 TRE, a MUC1/DF3 TRE, a TK TRE, a p21 TRE, a cyclin TRE, an HKLK2 TRE, a uPA TRE, a HER-2neu TRE, a prostate specific antigen (PSA) TRE, and a probasin TRE.

8. The HeLa-S3 cell of claim 5, wherein said coding region that is operatively linked to said TRE is selected from the group consisting of E1a, E1b, E2a, E2b and E4 coding regions.

9. The HeLa-S3 cell of claim 8, wherein said coding region is an E1a coding region.

10. The HeLa-S3 cell of claim 8, wherein said coding region is an E1b coding region.

11. The HeLa-S3 cell of claim 8, wherein said coding region is an E2a coding region.

12. The HeLa-S3 cell of claim 8, wherein said coding region is an E2b coding region.

13. The HeLa-S3 cell of claim 8, wherein said coding region is an E4 coding region.

14. The HeLa-S3 cell of claim 5, wherein said vector further comprises a second heterologous TRE operatively linked to the coding region of a second gene that is essential for replication of said vector, wherein said second TRE functions in said cell so that replication of the vector occurs in said cell.

15. The HeLa-S3 cell of claim 14, wherein the first and second heterologous TRE sequences are different.

16. The HeLa-S3 cell of claim 5, wherein said vector further comprises a heterologous gene.

17. The HeLa-S3 cell of claim 16, wherein said heterologous gene encodes GM-CSF.

18. A producer cell line comprising the cell of claim 1.

19. A producer cell line comprising the cell of claim 5.

20. A method of producing a replication-competent adenovirus, comprising culturing the HeLa-S3 cell of claim 1 and recovering said adenovirus from said cell or the supernatant of said cell.

21. A method of producing a replication-competent adenovirus, comprising culturing the HeLa-S3 cell of claim 5 and recovering said adenovirus from said cell or the supernatant of said cell.

22. The method according to claim 21, wherein said TRE comprises a promoter or enhancer.

23. The method according to claim 22, wherein said TRE is selected from the group consisting of an E2F-responsive TRE, a human telomerase reverse transcriptase (hTERT) TRE, an osteocalcin TRE, a carcinoembryonic antigen (CEA) TRE, a DF3 TRE, an α-fetoprotein TRE, an ErbB2 TRE, a surfactant TRE, a tyrosinase TRE, a PRL-3 TRE, a MUC1/DF3 TRE, a TK TRE, a p21 TRE, a cyclin TRE, an HKLK2 TRE, a uPA TRE, a HER-2neu TRE, a prostate specific antigen (PSA) TRE, and a probasin TRE.

24. The method according to claim 21, wherein said coding region that is operatively linked to said TRE is selected from the group consisting of E1a, E1b, E2a, E2b and E4 coding regions.

25. The method according to claim 24, wherein said vector further comprises a heterologous gene.

26. The method according to claim 25, wherein said heterologous gene encodes GM-CSF.
